(19) United States
(12) Patent Application Publication
(10) Pub. No.: US 2020/0340183 A1
(43) Pub. Date: Oct. 29, 2020

(54) FILMS OF MICROFIBRILLATED CELLULOSE

(71) Applicant: Stora Enso OYJ, Helsinki (FI)

(72) Inventors: Gisela Cunha, Nacka (SE); Heidi Saxell, Stockholm (SE)

(73) Assignee: Stora Enso OYJ, Helsinki (FI)

(21) Appl. No.: 16/955,916

(22) PCT Filed: Dec. 20, 2018
(86) PCT No.: PCT/IB2018/060415

§ 371 Date: Jun. 19, 2020

(30) Foreign Application Priority Data
Dec. 21, 2017 (SE) 1751615-4

(51) Int. Cl. D21H 11/20; D21H 11/18; D21C 9/00; D21H 27/00; A61L 15/28

(52) U.S. Cl. CPC D21H 11/20; D21H 11/18; A61L 15/28; D21C 9/005; D21H 27/002; D21C 9/004

(57) ABSTRACT

A method is provided for preparing a film of crosslinked microfibrillated cellulose. Phosphorylated microfibrillated cellulose is cast or wet-laid into a film; and then said film is post-treated (e.g. by heat-treatment) to provide crosslinking between the phosphorylated microfibrillated cellulose. Films and hygiene products comprising such films are also described.

A method for providing films of crosslinked microfibrillated cellulose is provided, as well as cast or wet-laid films of crosslinked phosphorylated microfibrillated cellulose. Products comprising said films are also described. Such films exhibit desirable properties, e.g. strength (in particular wet-strength), water absorbance and elasticity/flexibility.

### BACKGROUND

Microfibrillated cellulose (MFC) comprises partly or totally fibrillated cellulose or lignocellulose fibers. The liberated fibrils have a diameter less than 100 nm, whereas the actual fibril diameter or particle size distribution and/or aspect ratio (length/width) depends on the source and the manufacturing methods. The smallest fibril is called elementary fibril and has a diameter of approximately 2-4 nm (see e.g. Chinga-Carrasco, G., Nanoscale research letters 2011, 6:417), while it is common that the aggregated form of the elementary fibrils, also defined as microfibril, is the main product that is obtained when making MFC e.g. by using an extended refining process or pressure-drop disintegration process (see Fengel, D., Tappi J., March 1970, Vol 53, No. 3.). Depending on the source and the manufacturing process, the length of the fibrils can vary from around 1 to more than 10 micrometers. A coarse MFC grade might contain a substantial fraction of fibrillated fibers, i.e. protruding fibrils from the tracheid (cellulose fiber), and with a certain amount of fibrils liberated from the tracheid (cellulose fiber).

There are different acronyms for MFC such as cellulose microfibrils, fibrillated cellulose, nanofibrillated cellulose, fibril aggregates, nanoscale cellulose fibrils, cellulose nanofibers, cellulose nanofibrils, cellulose microfibers, cellulose fibrils, microfibrillar cellulose, microfibril aggregrates and cellulose microfibril aggregates. MFC can also be characterized by various physical or physical-chemical properties such as large surface area or its ability to form a gel-like material at low solids (1-5 wt %) when dispersed in water.

MFC exhibits useful chemical and mechanical properties. Chemical surface modification of MFC has the potential to improve the properties of MFC itself, as well as films wet-laid or cast from MFC, e.g. mechanical strength, water absorbance and elasticity/flexibility.

An additional problem with chemically modified MFC is that it has increased water absorption when compared to non-modified MFC, due to its chemical charge, and can start losing integrity upon contact with water. A balance of mechanical strength and water absorbance can therefore be difficult to achieve.

Other documents in this technical field include U.S. Pat. Nos. 4,256,111 and 6,027,536.

There is therefore a need to improve the properties of films cast from MFC; in particular, (wet) strength, water absorption and elasticity/flexibility properties. Suitably, the improvement can be achieved in a straightforward manner, without the use of external modifiers such as crosslinkers.

### SUMMARY

It has been found by the present inventor(s) that fibrous materials (i.e. films) with desirable elasticity and water absorption can be formed from a cellulose composition comprising phosphorylated microfibrillated cellulose (P-MFC).

A method for preparing a film of crosslinked microfibrillated cellulose is thus provided, said method comprising the steps of:
i. casting or wet-laying a cellulose composition comprising phosphorylated microfibrillated cellulose (P-MFC); said cellulose composition additionally comprising unmodified microfibrillated cellulose and/or chemically-modified microfibrillated cellulose; into a film;
ii. heat-treatment of said film to provide crosslinking of the phosphorylated microfibrillated cellulose.

A cast film is also provided by the method set out herein. A water-absorbent material comprising the film is provided. In a further aspect, a hygiene product comprising the film and/or water-absorbent material is provided.

Further aspects of the invention are provided in the following text and in the dependent claims.

### DETAILED DISCLOSURE

In a first aspect, the invention provides a method for preparing a film of crosslinked microfibrillated cellulose (MFC).

Microfibrillated cellulose (MFC) or so called cellulose microfibrils (CMF) shall in the context of the patent application mean a nano-scale cellulose particle fiber or fibril with at least one dimension less than 100 nm. MFC comprises partly or totally fibrillated cellulose or lignocellulose fibers. The cellulose fiber is preferably fibrillated to such an extent that the final specific surface area of the formed MFC is from about 1 to about 300 m²/g, such as from 1 to 200 m²/g or more preferably 50-200 m²/g when determined for a freeze-dried material with the BET method.

Various methods exist to make MFC, such as single or multiple pass refining, pre-hydrolysis followed by refining or high shear disintegration or liberation of fibrils. One or several pre-treatment steps are usually required in order to make MFC manufacturing both energy efficient and sustainable. The cellulose fibers of the pulp to be supplied may thus be pre-treated enzymatically or chemically, for example to reduce the quantity of hemicellulose or lignin. The cellulose fibers may be chemically modified before fibrillation, wherein the cellulose molecules contain functional groups other (or more) than found in the original cellulose. Such groups include, among others, carboxymethyl (CMC), aldehyde and/or carboxyl groups (cellulose obtained by N-oxyl mediated oxidation, for example “TEMPO”), or quaternary ammonium (cationic cellulose). After being modified or oxidized in one of the above-described methods, it is easier to disintegrate the fibers into MFC or NFC.

The nanofibrillar cellulose may contain some hemicelluloses; the amount is dependent on the plant source. Mechanical disintegration of the pre-treated fibers, e.g. hydrolysed, pre-swelled, or oxidized cellulose raw material is carried out with suitable equipment such as a refiner, grinder, homogenizer, colloider, friction grinder, ultrasound sonicator, single or twin-screw extruder, fluidizer such as microfluidizer, macrofluidizer or fluidizer-type homogenizer. Depending on the MFC manufacturing method, the product might also contain fines, or nanocrystalline cellulose or e.g. other chemicals present in wood fibers or in papermaking process. The product might also contain various amounts of micron size fiber particles that have not been efficiently fibrillated.

MFC can be produced from wood cellulose fibers, both from hardwood or softwood fibers. It can also be made from microbial sources, agricultural fibers such as wheat straw pulp, bamboo, bagasse, or other non-wood fiber sources. It is preferably made from pulp including pulp from virgin fiber, e.g. mechanical, chemical and/or thermomechanical pulps. It can also be made from broke or recycled paper.

The above described definition of MFC includes, but is not limited to, the proposed TAPPI standard W13021 on cellulose nano or microfibril (CMF) defining a cellulose nanofiber material containing multiple elementary fibrils with both crystalline and amorphous regions, having a high aspect ratio with width of 5-30 nm and aspect ratio usually greater than 50.

Phosphorylated microfibrillated cellulose (P-MFC) is typically obtained by reacting cellulose pulp fibers with a phosphorylating agent such as phosphoric acid, and subsequently fibrillating the fibers to P-MFC. One particular method involves providing a suspension of cellulose pulp fibers in water, and phosphorylating the cellulose pulp fibers in said water suspension with a phosphorylating agent, followed by fibrillation with methods common in the art. Suitable phosphorylating agents include phosphoric acid, phosphorus pentaoxide, phosphorus oxychloride, diammonium hydrogen phosphate and sodium dihydrogen phosphate.

In the reaction to form P-MFC, alcohol functionalities (—OH) in the cellulose are converted to phosphate groups (—OPO₃²⁻). In this manner, crosslinkable functional groups (phosphate groups) are introduced to the pulp fibers or microfibrillated cellulose.

In a first general step of the method, cellulose composition comprising phosphorylated microfibrillated cellulose (P-MFC) is formed (e.g. wet-laid or cast) into a film.

In the case that the cellulose composition comprises P-MFC, components other than P-MFC may be present in the composition. However, the cellulose composition suitably comprises more than 25%, preferably more than 50%, such as e.g. more than 75% by weight P-MFC. The cellulose composition comprising P-MFC additionally comprises unmodified (native) MFC. By “unmodified” or “native” MFC is meant microfibrillated cellulose which is the direct result of fibrillation of native cellulose fibers, i.e. without chemical treatment before or after fibrillation.

Suitably, therefore, the cellulose composition consists of P-MFC and MFC. Alternatively or additionally, the cellulose composition comprising P-MFC additionally comprises chemically-modified microfibrillated cellulose, such as e.g. dialdehyde-MFC or TEMPO-MFC (i.e. MFC oxidised with 2,2,6,6-tetramethylpiperidin-1-yl)oxidanyl). Additional components of the cellulose composition may include natural or synthetic filaments or natural or synthetic staple fibres.

In a second general step of the method, the film from the first step is heat-treated so as to provide crosslinking of the phosphorylated microfibrillated cellulose. Crosslinking suitably takes place without the use of any additional crosslinking agents; i.e. crosslinks are formed directly between the phosphate moieties and other components of the cellulose composition.

Heat treatment in the second general step of the method suitably takes place at a temperature of between 60 and 200° C., e.g. between 70 and 120° C. Such temperatures are sufficient to obtain crosslinking, but also limit potential degradation of the MFC. It has been established that heat treatment suitably takes place for a time of between 10 and 180 minutes, depending on the temperature used and initial solids content of the material to heat treat. Heat treatment may take place in an oven, but other methods of heat treatment may also be used.

The fibrous material is a film, and the composition is cast or wet-laid using techniques common in the art. The present technology provides the possibility of creating P-MFC films with water absorption capacity, elasticity and wet-strength. Such films could be used to prepare moulded materials with different shapes upon drying the wetted crosslinked films in different molds.

The general steps of the method (forming, followed by heat-treatment) may be carried out without any intervening method steps. Alternatively, one or more intervening method steps may be carried out between the forming step and the heat-treatment step. In one particular aspect, the film may be dried before or during the heat-treatment step. Drying can suitably take place under ambient conditions (e.g. 25° C.). It has been discovered that crosslinking can be triggered in film which has been previously dried at ambient conditions, e.g. by putting dried film according to the invention in the oven. This means that one can in principle dry the material at ambient conditions (with no crosslinking) and then trigger the crosslinking when desired at a later stage by heat-treatment.

Alternatively, the step of drying the film can take place during the heat-treatment step. In this alternative, a dry, crosslinked film is obtained, which can have advantageous water-absorptive and strength properties both in dry and wet conditions.

If hydrated film is required, a further step of hydrating said film with water after the heat-treatment step may be carried out.

It is thought not to be enough to remove the water from the sample at room temperature (i.e. to dry at RT); a heat-treatment is required for the crosslinking. Furthermore, it was considered surprising that some stretchability/elasticity behaviour could be obtained after soaking the heat-treated material in water.

The presence of phosphate crosslinks between MFC fibrils can be ascertained by spectroscopic methods, e.g. ³¹P NMR.

In a similar manner, a cast film obtained via the method described herein is provided, as well as a cast film of crosslinked phosphorylated microfibrillated cellulose per se.

A hygiene product is provided which comprises the film of the invention and/or a water-absorbent material comprising said film. The hygiene product may be selected from the group consisting of a disposable diaper, a sanitary napkin, a wipe, a tampon, an absorbent dressing and a disposable tissue. A method for providing a hygiene product is also provided, said method comprising preparing a film of crosslinked phosphorylated microfibrillated cellulose according to the invention, and; incorporating said film into a hygiene product. The skilled person is aware of standard methods for constructing hygiene products, and incorporating fibrous material into such products.

### Examples

1. Preparation of a P-MFC Cast Film

### Materials:

1. P-MFC 2: DS=0.86 mmol/g; pH=9.3; 1.5 wt %

A P-MFC film was cast, and subsequently heat-treated in the oven at 105° C. for 20 min. It possessed wet-strength, flexibility and even a slight elasticity in wet-state (assessed manually).

2. Swelling of P-MFC Films and Native MFC Films

### Materials:

1. P-MFC 2: DS=0.86 mmol/g; pH=9.3; 4.51 wt %
2. Native MFC: pH-4.18 wt %
3. TEMPO-MFC: DS=1.25 mmol/g; pH=10.5; 1.94 wt %

Two P-MFC films were cast by spreading P-MFC with a spatula on glass petri dishes. One film was left at ambient temperature overnight whereas the other one was first dried in the oven for 1 hour at 105° C. before it was left in room temperature overnight. The two films were then soaked in an excess of deionized water for 4.5 hours and their weight was measured again.

The same procedure was performed using native MFC and TEMPO-oxidized MFC (T-MFC).

| | | | | Swelling |
|---|---|---|---|---|
| | | | Weight after | degree (g |
| | Drying method | Dry weight (g) | swelling (g) | water/g) |
| P-MFC | 1 hour oven + | 0.20 | 3.68 | 18.4 |
| | ambient | | | |
| | overnight | | | |
| P-MFC | Ambient | 0.22 | 12.23 | 55.6 |
| | overnight | | | |
| Native | 1 hour oven + | 0.21 | 1.04 | 5.0 |
| | ambient | | | |
| | overnight | | | |
| Native | Ambient | 0.21 | 1.09 | 5.2 |
| | overnight | | | |
| T-MFC | 1 hour oven + | 0.24 | 0.47 | 2.0 |
| | ambient | | | |
| | overnight | | | |
| T-MFC | Ambient | 0.26 | 0.54 | 2.1 |
| | overnight | | | |

After soaking of the films in water, the oven-dried P-MFC film showed a good wet strength and some stretchability/elasticity, whereas ambient dried P-MFC and native samples were very fragile and fell apart if manually handled. The T-MFC samples had good integrity, both ambient- and oven-dried, but the swelling after drying was very low. The P-MFC had a much higher capacity in terms of water absorbance than the other samples regardless of drying method, although it is apparent that it swells less after oven drying.

### ADDITIONAL CONCLUSIONS

One P-MFC film was made with the same procedure as above, but taken out of the oven after 30 minutes, before it was completely dry. After drying completely at room temperature, it was soaked in water. The dry/wet strength seemed as poor as for the films only dried at room temperature.
One P-MFC film that had been dried in ambient atmosphere was put in the oven for 30 minutes before it was soaked in water, and exhibited dry/wet strength.

1. A method for preparing a film of crosslinked microfibrillated cellulose, said method comprising the steps of:
i. casting or wet-laying a cellulose composition comprising phosphorylated microfibrillated cellulose (P-MFC) into a film, said cellulose composition additionally comprising unmodified microfibrillated cellulose, chemically-modified microfibrillated cellulose, or both; and,
ii. heat-treatment of said film to provide crosslinking of the phosphorylated microfibrillated cellulose.

2. The method according to claim 1, wherein said cellulose composition comprises unmodified microfibrillated cellulose.

3. The method according to claim 1, wherein said cellulose composition comprises chemically-modified microfibrillated cellulose.

4. The method according to claim 1, wherein said cellulose composition comprises more than 25% by weight P-MFC.

5. The method according to claim 1, wherein said heat treatment takes place at a temperature of between 60 and 200° C.

6. The method according to claim 1, wherein said heat treatment takes place for a time of between 10 and 180 minutes.

7. The method according to claim 1, wherein the crosslinking takes place in the absence of any additional crosslinking agents.

8. The method according to claim 1, wherein said phosphorylated microfibrillated cellulose (P-MFC) is obtained by:
providing a suspension of cellulose pulp fibers in water, and
phosphorylating the cellulose pulp fibers in said water suspension with a phosphorylating agent, followed by fibrillation.

9. The method according to claim 1, further comprising the step of drying said film, before the heat-treatment step.

10. The method according to claim 1, further comprising the step of hydrating said film with water after the heat-treatment step.

11. A cast or wet-laid film obtained via the method of claim 1.

12. A cast or wet-laid film comprising crosslinked phosphorylated microfibrillated cellulose.

13. A water-absorbent material comprising the film according to claim 11.

14. A hygiene product comprising the film according to claim 11.

15. The hygiene product according to claim 14, wherein the hygiene product is selected from the group consisting of a disposable diaper, a sanitary napkin, a wipe, a tampon, an absorbent dressing and a disposable tissue.

16. A method for providing a hygiene product, said method comprising:
preparing a film of crosslinked microfibrillated cellulose according to claim 1, and;
incorporating said film into a hygiene product.

17. The method according to claim 1, wherein said heat treatment takes place at a temperature of between 70 and 120° C.

18. The method according to claim 1, wherein said cellulose composition comprises more than 50% by weight P-MFC.

19. The method according to claim 1, wherein said cellulose composition comprises more than 75% by weight P-MFC.
